(19) United States
(12) Patent Application Publication
(10) Pub. No.: US 2021/0378582 A1
(43) Pub. Date: Dec. 9, 2021

(54) SYSTEMS AND METHODS FOR ASSESSING STROKE RISK

(71) Applicant: Covidien LP, Mansfield, MA (US)

(72) Inventors: Rachel Day, Tempe, AZ (US); Fatmah Mouslli, Phoenix, AZ (US); Michael Ferguson, Gilbert, AZ (US); Emily Byrne, Tempe, AZ (US); Sean Allen, Tempe, AZ (US); Naisargi Nandedkar, Chandler, AZ (US)

(21) Appl. No.: 17/303,701

(22) Filed: Jun. 4, 2021

Related U.S. Application Data
(60) Provisional application No. 62/705,029, filed on Jun. 8, 2020.

(51) Int. Cl. A61B 5/378; A61B 5/38; A61B 5/384; A61B 5/291; A61B 5/02; A61B 5/00; G06F 3/01

(52) U.S. Cl. CPC A61B 5/378; A61B 5/38; A61B 5/384; G06F 3/011; A61B 5/02042; A61B 5/7275; A61B 5/746; A61B 5/291

(57) ABSTRACT

A system for assessing stroke conditions includes a wearable stimulator and a sensor device configured to obtain physiological data from a patient. The sensor device can include electrodes configured to detect electrical signals corresponding to brain activity. A computing device communicatively coupled to the wearable stimulator and the sensor device is configured to receive the physiological data and analyze the physiological data to provide a patient stroke assessment.

### CROSS-REFERENCE TO RELATED APPLICATION(S)

The present application claims the benefit of priority to U.S. Provisional Patent Application No. 62/705,029, filed Jun. 8, 2020, which is incorporated by reference herein in its entirety.

### TECHNICAL FIELD

The present technology is directed to medical devices and, more particularly, to systems and methods for assessing stroke risk.

### BACKGROUND

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism. During a stroke, the blood supply to an area of a brain may be decreased, which can lead to dysfunction of the brain tissue in that area.

Stroke is the number two cause of death worldwide and the number one cause of disability. Speed to treatment is the critical factor in stroke treatment as 1.9M neurons are lost per minute on average during stroke. Stroke diagnosis and time between event and therapy delivery are the primary barriers to improving therapy effectiveness. Stroke has 3 primary etiologies; i) ischemic stroke (representing about 65% of all strokes), ii) hemorrhagic stroke (representing about 10% of all strokes), and iii) cryptogenic strokes (includes TIA, representing 25% of all strokes). Strokes can be considered as having neurogenic and/or cardiogenic origins.

A variety of approaches exist for treating patients undergoing a stroke. For example, a clinician may administer anticoagulants, such as warfarin, or may undertake intravascular interventions such as thrombectomy procedures to treat ischemic stroke. For example, a clinician may administer antihypertensive drugs, such as beta blockers (e.g., Labetalol) and ACE-inhibitors (e.g., Enalapril) or may undertake intravascular interventions such as coil embolization to treat hemorrhagic stroke. Lastly, if stroke symptoms have resolved on their own with negative neurological work-up, a clinician may administer long-term cardiac monitoring (external or implantable) to determine potential cardiac origins of cryptogenic stroke. However, such treatments may be frequently underutilized and/or relatively ineffective due to the failure to timely identify whether a patient is undergoing or has recently undergone a stroke. This is a particular risk with more minor strokes that leave patients relatively functional upon cursory evaluation.

### SUMMARY

The present technology is illustrated, for example, according to various aspects described below. Various examples of aspects of the present technology are described as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.

The present technology can include a system for assessing stroke conditions comprising a sensor configured to receive physiological data from a patient, a wearable stimulator configured to generate a stimulus, and a computing device communicatively coupled to the sensor and the wearable stimulator. The computing device can be configured to cause the stimulator to output a stimulus, receive the physiological data from the sensor, analyze the physiological data and, based on the analysis, provide a patient stroke assessment.

In some embodiments, the computing device is configured to output a visual stimulus and/or an audio stimulus via the wearable stimulator. In several of such embodiments, each of the visual stimulus and the audio stimulus is configured to stimulate a stroke event for the patient.

According to some embodiments, the stimulator is a wearable display. In several embodiments, the stimulator is a virtual reality (VR) headset.

In some embodiments, the physiological data comprises brain activity data. In some embodiments, the sensor comprises a plurality of electrodes configured to detect brain activity data.

In several embodiments, the sensor comprises a plurality of electroencephalogram (EEG) electrodes.

Providing the patient stroke assessment can include one, some, or all of: classifying an identified stroke as ischemic or hemorrhagic, determining whether a patient has suffered a stroke, determining a risk that a patient will suffer a stroke, providing a confidence score associated with a determination of patient stroke, providing recommended therapeutic action accompanying a stroke determination, or transmitting an alert to an emergency healthcare provider.

According to several embodiments, the sensor is configured to be disposed at or adjacent a rear portion of the patient's neck or skull base. In some embodiments, the sensor is configured to be disposed above the patient's shoulders. In some embodiments, the sensor is configured to be disposed at or below the patient's occipital bone. In some embodiments, the sensor comprises a housing configured to be implanted within the patient. In several of such embodiments, the housing is configured to be implanted subcutaneously. In some embodiments, the sensor comprises a housing configured to be disposed over the patient's skin. In some embodiments, the sensor device includes electrodes configured to contact the patient's skin. In some embodiments, the electrodes include protrusions configured to at least partially penetrate the patient's skin. In several of such embodiments, the protrusions comprise microneedles.

In some embodiments, the sensor comprises an EEG array. The EEG array, for example, can comprise at least 2 electrodes, at least 3 electrodes, at least 4 electrodes, at least 5 electrodes, fewer than 6 electrodes, fewer than 5 electrodes, fewer than 4 electrodes, or fewer than 3 electrodes.

In some embodiments, the sensor can comprise a housing having a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc.

In some embodiments, the sensor device and the computing device are enclosed within a common housing.

According to several aspects of the technology, the physiological data comprises at least three channels of EEG signals. In some embodiments, the physiological data comprises brain activity data. In some embodiments, the physiological data comprises electrical brain activity data and electrical heart activity data, and analyzing the physiological data comprises filtering the physiological data to separate the electrical brain activity data from the electrical heart activity data. In some embodiments, the physiological data comprises electrical signals detected via electrodes of the sensor device, and analyzing the physiological data comprises analyzing the electrical signals to detect brain activity. In some embodiments, analyzing the electrical signals to detect brain activity data comprises filtering the electrical signals to reduce a contribution of electrical signals generated from heart activity. In some embodiments, analyzing the electrical signals to detect brain activity data comprises filtering the electrical signals to reduce a contribution of electrical signals generated from muscle activity. In some embodiments, the physiological data comprises motion data. The physiological data can include any combination of the foregoing parameters and analysis.

Various aspects of the technology include a method for assessing stroke conditions comprising outputting a stimulus to a patient via a wearable stimulator, receiving physiological data from a sensor configured to obtain the physiological data from the patient, analyzing the physiological data, and, based on the analysis, providing a patient stroke assessment.

In some embodiments of the methods herein, the computing device is configured to output a visual stimulus and/or an audio stimulus via the wearable stimulator. In several of such embodiments, each of the visual stimulus and the audio stimulus is configured to stimulate a stroke event for the patient.

In several embodiments of the methods herein, the stimulator is a wearable display. In some embodiments, the stimulator is a virtual reality (VR) headset.

In some embodiments, the stimulus is a first stimulus and the method further comprises outputting a second stimulus after the first stimulus based on the analysis. The second stimulus can be the same or different than the first stimulus.

According to some embodiments of the methods herein, the physiological data comprises brain activity data.

In some embodiments of the methods herein, the sensor comprises a plurality of electrodes configured to detect brain activity data.

In some embodiments, the sensor comprises a plurality of electroencephalogram (EEG) electrodes.

Providing the patient stroke assessment can include one, some, or all of: classifying an identified stroke as ischemic or hemorrhagic, determining whether a patient has suffered a stroke, determining a risk that a patient will suffer a stroke, providing a confidence score associated with a determination of patient stroke, providing recommended therapeutic action accompanying a stroke determination, or transmitting an alert to an emergency healthcare provider.

In several embodiments, the sensor is configured to be disposed at or adjacent a rear portion of the patient's neck or skull base. In some embodiments of the methods herein, the sensor is configured to be disposed above the patient's shoulders. In some embodiments of the methods herein, the sensor is configured to be disposed at or below the patient's occipital bone.

In some embodiments of the methods herein, the sensor comprises a housing configured to be implanted within the patient. For example, the housing can be configured to be implanted subcutaneously. In some embodiments, the sensor comprises a housing configured to be disposed over the patient's skin. In some embodiments, the sensor device includes electrodes configured to contact the patient's skin. For example, the electrodes can be placed on the surface of the patient's skin. In some embodiments, the electrodes include protrusions configured to at least partially penetrate the patient's skin. The protrusions can comprise microneedles or other penetrating members. In some embodiments, the sensor comprises an EEG array. The EEG array, for example, can comprise at least 2 electrodes, at least 3 electrodes, at least 4 electrodes, at least 5 electrodes, fewer than 6 electrodes, fewer than 5 electrodes, fewer than 4 electrodes, or fewer than 3 electrodes. In some embodiments of the methods herein, the sensor comprises a housing having a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc.

In some embodiments of the methods herein, the sensor device and the computing device are enclosed within a common housing.

According to several aspects of the methods herein, the physiological data comprises at least three channels of EEG signals. In some embodiments, the physiological data comprises electrical brain activity data and electrical heart activity data, and analyzing the physiological data comprises filtering the physiological data to separate the electrical brain activity data from the electrical heart activity data. In some embodiments, the physiological data comprises electrical signals detected via electrodes of the sensor device, and analyzing the physiological data comprises analyzing the electrical signals to detect brain activity. In some embodiments, analyzing the electrical signals to detect brain activity data comprises filtering the electrical signals to reduce a contribution of electrical signals generated from heart activity. In some embodiments, analyzing the electrical signals to detect brain activity data comprises filtering the electrical signals to reduce a contribution of electrical signals generated from muscle activity. In some embodiments, the physiological data comprises motion data. The physiological data can include any combination of the foregoing parameters and analysis.

Additional features and advantages of the present technology will be set forth in the description below, and in part will be apparent from the description, or may be learned by practice of the subject technology. The advantages of the present technology will be realized and attained by the structure particularly pointed out in the written description and claims hereof as well as the appended drawings.

It is to be understood that both the foregoing general description and the following detailed description are exemplary and explanatory and are intended to provide further explanation of the present technology as claimed.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is a schematic diagram of a stroke assessment system configured in accordance with embodiments of the present technology.

FIG. 2 depicts a patient wearing a wearable stimulator in accordance with embodiments of the present technology.

FIG. 3 is a flow diagram of another method for making a stroke determination in accordance with embodiments of the present technology.

### DETAILED DESCRIPTION

It can be difficult to determine whether a patient is suffering from a stroke, has suffered from a stroke, or is at high risk for suffering a stroke. Current diagnostic techniques typically involve evaluating a patient for visible symptoms, such as paralysis or numbness of the face, arm, or leg, as well as difficultly walking, speaking, or understanding. However, these techniques may result in undiagnosed strokes, particularly more minor strokes that leave patients relatively functional upon cursory evaluation. Even for relatively minor strokes, it is important to treat the patient as soon as possible because treatment outcomes for stroke patients are highly time-dependent. Accordingly, there is a need for improved methods for assessing strokes.

Embodiments of the present technology enable improved stroke risk assessment by utilizing augmented and/or virtual reality devices to stimulate the patient while employing a sensor device to monitor one or more physiological parameters. The sensor device, for example, can be equipped with electrodes (e.g., EEG electrodes) that can be used to sense and record a patient's brain electrical activity. The physiological data can be analyzed to provide a stroke assessment, as described in more detail below.

In some embodiments, the sensor data can be analyzed to make a stroke determination includes using a classification algorithm, which can itself be derived using machine learning techniques applied to databases of known stroke patient data. The detection algorithm(s) can be passive (involving measurement of a purely resting patient) or active (involving prompting a patient to perform potentially impaired functionality, such as moving particular muscle groups (e.g., raising an arm, moving a finger, moving facial muscles, etc.,) and/or speaking while recording the electrical response).

### Example Systems

The following discussion provides a brief, general description of a suitable environment in which the present technology may be implemented. Although not required, aspects of the technology are described in the general context of computer-executable instructions, such as routines executed by a general-purpose computer. Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth network) on a propagated signal on a propagation medium (e.g., an electromagnetic wave(s), a sound wave) over a period of time, or may be provided on any analog or digital network (packet switched, circuit switched, or other scheme).

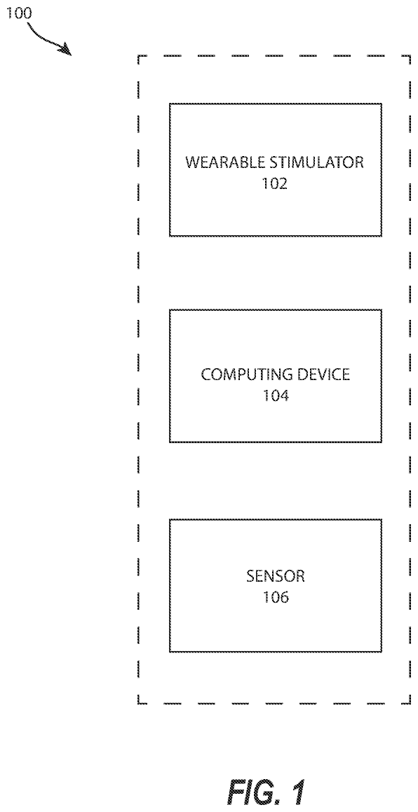

FIG. 1 shows a system 100 for stroke assessment configured in accordance with embodiments of the present technology. The system 100 may comprise a wearable stimulator 102, a computing device 104, and a sensor device 106. All or a portion of each of the computing device 104 and the sensor device 106 may be incorporated with the stimulator or may be a component separate from the stimulator 102. Although the system 100 is shown with certain devices for purposes of explanation, in various examples any one or more of the devices shown in FIG. 1 can be omitted. Similarly, although the devices shown in FIG. 1 are illustrated as including certain components, in various examples any one or more of the particular components within these devices can be omitted. For example, in some embodiments the stimulator device 100 may not fully cover the patient's eyes. Moreover, any of the devices can include additional components not specifically shown here.

The stimulator 102 can be configured to output a stimulus configured to trigger a stroke event, as measured by the physiological data (such as brain activity) monitored by the sensor device 106. For example, specific wavelengths in the brain can indicate abnormalities representative of a stroke. The stimulator 102 may work in conjunction with EEG monitoring and apply images and/or sound to stimulate an event. The usage of visuals and sounds will either increase or decrease the EEG readings, providing a direction for what symptoms are occurring and what treatment methods should be implemented.

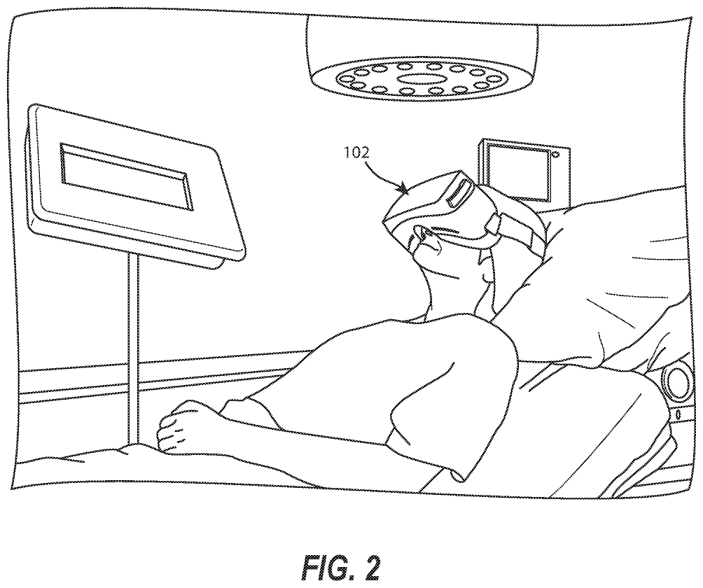

In some embodiments, for example as shown in FIG. 2, the wearable stimulator 102 may comprise a VR headset. The VR headset may include a display and a speaker. In some embodiments, the speaker is separate from the display. In several embodiments, the wearable stimulator 102 is an augmented reality (AR) headset.

In some embodiments, the system 100 administers different sets of visuals and/or sounds and analyzes the response. The system 100 may continually or intermittently take the EEG readings and adjust the visuals and sounds to understand the patient triggers. The current method for triggering an event can span across 12 hours and include one or more activities. In some embodiments, the patient may experience multiple different stimuli, such as increased visual/screen time, strobe lighting, and loud humming noises. The present technology decreases the time required to understand the patient's stroke triggers and thus reduces the risk of side effects, such as a migraine.

As previously mentioned, the system 100 may include a sensor device 106 configured to sense physiological patient data used by the computing device 104 to make a stroke assessment. In some embodiments, the sensor device 106 is configured to be implanted in a target site of the patient or disposed over the skin of the patient at a target site. The sensor device 106 may be a single sensing or a plurality of sensing devices. The sensor device 106 may be a relatively small device, and may be placed (e.g., inserted) under or over the skin at any location on the patient's body. As described in more detail below, the sensor device 106 can detect one more physiological parameters of a patient (e.g., electrical activity corresponding to brain activity in particular regions of the patient's brain, heart rhythm data, motion data, etc.).

The stimulator 102 and the sensor device 106 can be communicatively coupled to the computing device 104 via a wired or wireless connection. In some embodiments, the computing device 104 can be integrated with one or both of the stimulator 102 and the sensor device 106. In some embodiments, the computing device 104 may be a separate component, such as a mobile device (e.g., a smartphone, tablet, smartwatch, etc.) or other computing device controlled by the clinician. In operation, the patient, stimulator 102, and/or sensor device 106 may receive output or instructions from the computing device 104 that are based at least in part on data received at the computing device 104 from the sensor device 106. For example, the computing device 104 may adjust the type, frequency, and/or duration of the stimulus based on a brain activity measurement obtained by the sensor device 106.

In some embodiments, the computing device 104 outputs user prompts which can be synchronized with data collection via the sensor device 106. For example, the computing device 104 may instruct the user to lift an arm, make a facial expression, etc., and the sensor device 106 may record physiological data while the user performs the requested actions. Moreover, the computing device 104 may itself analyze the patient (e.g., the patient's activity or condition in response to such prompts), for example using a camera to detect facial drooping, using a microphone to detect slurred speech, or to detect any other indicia of stroke. In some embodiments, such indicia can be compared against pre-stroke inputs (e.g., a stored baseline facial image or voice-print with baseline speech recording).

The sensor device 106 and/or the computing device 104 can also be communicatively coupled with one or more external computing devices (e.g., over a wide area network and/or a local area network). In some examples, the external computing devices can take the form of servers, personal computers, tablet computers or other computing devices associated with one or more healthcare providers (e.g., hospitals, medical data analytic companies, device manufacturers, etc.). These external computing devices can collect data recorded by the sensor device 106, the computing device 104, and/or the stimulator 102. In some embodiments, such data can be anonymized and aggregated to perform large-scale analysis (e.g., using machine-learning techniques or other suitable data analysis techniques) to develop and improve stroke detection algorithms using data collected by a large number of sensor devices 106. Additionally, the external computing devices may transmit data to the computing device 104, the stimulator 102, and/or the sensor device 106. For example, an updated algorithm for making stroke determinations may be developed by the external computing devices (e.g., using machine learning or other techniques) and then provided to the sensor device 106, stimulator 102, and/or the computing device 104 via the network (e.g., as an over-the-air update), and installed on the sensor device 106, the stimulator 102, and/or the computing device 104.

In some embodiments, the system 100 can also include additional implantable devices, such as an implantable cardiac monitors, an implantable pacemaker, an implantable cardiac defibrillator, a cardiac resynchronization therapy (CRT) device (e.g., CRT-D defibrillator or CRT-P pacemaker), a neurostimulator, a deep-brain stimulation device, a nerve stimulator, a drug pump (e.g., an insulin pump), a glucose monitor, or other devices. Other devices that may support and enhance a personal ecosystem to reduce stroke risk include fitness monitors, nutrition devices, etc. Additionally or alternatively, a stroke detection device can be used in conjunction with other disease therapies with high risk of stroke as an adverse event (e.g., LVAD devices, TAVI/TAMR devices, bariatric/gastric surgery, etc.).

As noted previously, the sensor device 106 is configured to be coupled to a patient for recording physiological data relevant to a stroke assessment. For example, the sensor device 106 can be implanted within the body of a patient, may be disposed directly over a patient's skin (e.g., held in place via an adhesive or fastener), or may be removably worn by the patient. The sensor device 106 may include sensing components, which can include a number of different sensors and/or types of sensors. For example, the sensing components can include a plurality of electrodes, an accelerometer, and optionally other sensors. Examples of other sensors include a blood pressure sensor, a pulse oximeter, an ECG sensor or other heart-recording device, an EMG sensor or other muscle-activity recording device, a temperature sensor, a skin galvanometer, hygrometer, altimeter, gyroscope, magnetometer, proximity sensor, hall effect sensors, or any other suitable sensor for monitoring physiological characteristics of the patient. These particular sensing components are exemplary, and in various embodiments the sensors employed can vary.

The electrodes can be configured to detect electrical activity such as brain activity (e.g., EEG data), heart activity (e.g., ECG data), and/or muscle activity (e.g., EMG data). The electrodes may be formed from any suitable conductive material or materials to enable the electrodes to perform electrical measurements on the patient. In some embodiments, the sensor device 106 can be configured to analyze data from the electrodes to extract both brain activity data (e.g., EEG signals) and heart activity data (e.g., ECG signals). The brain activity data may be evaluated to provide a stroke determination or other assessment of brain condition, while the heart activity data may be evaluated to provide an assessment of heart condition or to detect certain cardiac events (e.g., heart rate variability, arrhythmias (e.g., tachyarrhythmias or bradycardia), ventricular or atrial fibrillation episodes, etc.).

In some embodiments, the computing device 104 and/or sensor device 106 is configured to analyze data from the electrodes to extract brain activity data and to discard or reduce any contribution from heart or muscle activity. In some embodiments, the electrodes are configured to be disposed over the patient's skin. In such embodiments, the electrodes can include protrusions (e.g., microneedles or other suitable structures) configured to at least partially penetrate the patient's skin so as to improve detection of subcutaneous electrical activity. In some embodiments, the sensor device 106 can be configured to be implanted within the body (e.g., subcutaneously), and as such the electrodes can include a conductive surface exposed along at least a portion of the sensor device 106 so as to detect electrical activity within the body.

The computing device 104 and/or sensor device 106 may be configured to calculate physiological characteristics relating to one or more electrical signals received from the electrodes. For example, the computing device 104 and/or sensor device 106 may be configured to algorithmically determine the presence or absence of a stroke or other neurological condition from the electrical signal. In certain embodiments, the computing device 104 and/or sensor device 106 may make a stroke assessment for each electrode (e.g., channel) or may make a stroke assessment using electrical signals acquired from two or more selected electrodes.

In various embodiments, the number and configuration of electrodes can vary. For example, the sensor device 106 can include at least 2, at least 3, at least 4, at least 5, or more electrodes in an array. In some embodiments, the sensor device 106 includes fewer than 6, fewer than 5, fewer than 4, or fewer than 3 electrodes in an array. As described in more detail below, although conventional EEG arrays include a large number of electrodes disposed over the top of a patient's head, some embodiments of the present technology include a relatively small number of electrodes (e.g., three electrodes) configured to be placed over the back of the patient's neck or base of the skull. In this position, electrical data collected via these electrodes may correspond to brain activity in regions determined to be of interest for stroke determination (e.g., the P3, Pz, and/or P4 regions).

In some embodiments, the electrodes may all reside within a single housing of the sensor device 106. In some embodiments, the electrodes may extend away from a housing of the sensor device 106 and be connected via leads or other connective components. For example, the sensor device 106 can include a housing that encompasses certain components (e.g., power, a communications link, processing circuitry, and/or memory), and the electrodes (and/or other sensing components) can be coupled to the housing via electrical leads or other suitable connections. In such configurations, the electrodes can be positioned at locations spaced apart from the housing of the sensor device 106. In some embodiments, the electrodes can be disposed within discrete housings that are in turn coupled to a housing containing the other components of the sensor device 106. Such a configuration, in which multiple housings (or sub-housings) are coupled together via flexible or other connectors, may facilitate placement of the sensor device 106 at a desired location to improve patient comfort. Additionally, this may facilitate placement of electrodes at desirable positions for detecting clinically useful brain activity data.

The accelerometer can be configured to detect patient movement and, in some embodiments, the sensor device 106 can be configured to initiate monitoring of brain activity via the electrodes upon certain movement detection using the accelerometer. In some embodiments, the sensing performed via the electrodes can be modified in response to a particular movement, for example with an increased sampling rate or other modification.

The sensor device 106 can also include a power source (e.g., a battery, capacitors). In some embodiments, the power source can be rechargeable, for example using inductive charging or other wireless charging techniques. Such rechargeability can facilitate long-term placement of the sensor device on or within a patient.

The wearable stimulator, the sensor device, and/or the computing device may include a communications link that enables transmission of data and/or receipt of data from external devices (e.g., such as an external computing device). The communications link can include a wired communication link and/or a wireless communication link (e.g., Bluetooth, Near-Field Communications, LTE, 5G, Wi-Fi, infrared and/or another wireless radio transmission network).

The processing circuitry can include one or more CPUs, ASICs, digital signal processing circuitry, or any other suitable electrical components configured to process data from the sensing components and control operation of the sensor device 106. In some embodiments, the processing circuitry includes hardware particularly adapted for artificial intelligence and/or machine learning applications, for example, a tensor processing unit (TPU) or other such hardware. In certain embodiments, the processing circuitry of the sensor device 106 may include one or more input protection circuits to filter the electrical signals and may include amplifier/filter circuitry to remove DC and high frequency components, one or more analog-to-digital (A/D) converters, or any other suitable components.

The sensor device 106 can further include memory, which can take the form of one or more computer readable storage modules configured to store information (e.g., signal data, subject information or profiles, environmental data, data collected from one or more sensing components, media files) and/or executable instructions that can be executed by the processing circuitry. The memory can include, for example, instructions for analyzing patient data to determine whether a patient is undergoing or has recently or previously undergone a stroke. In some embodiments, the memory stores data (e.g., signal data acquired from the sensing components) used in the stroke detection techniques disclosed herein.

As noted above, in some embodiments, the sensor device 106 may communicate with the computing device 104. The computing device 104 can be, for example, a smartwatch, smartphone, laptop, tablet, desktop PC, or any other suitable computing device and can include one or more features, applications and/or other elements commonly found in such devices. For example, the computing device 104 can include display, a communications link (e.g., a wireless transceiver that may include one or more antennas for wirelessly communicating with, for example, other devices, websites, and the sensor device 106). Communication between the computing device 104 and other devices can be performed via, e.g., a network (which can include the Internet, public and private intranet, a local or extended Wi-Fi network, cell towers, the plain old telephone system (POTS), etc.), direct wireless communication, etc. The computing device 104 can additionally include well-known input components and output components, including, for example, a touch screen, a keypad, speakers, a camera, etc.

As noted above, in some embodiments, the sensor device 106 may communicate with the stimulator 102. The stimulator 102 can be, for example, a smartwatch, smartphone, laptop, tablet, desktop PC, or any other suitable computing device and can include one or more features, applications and/or other elements commonly found in such devices. For example, the stimulator 102 can include display, a communications link (e.g., a wireless transceiver that may include one or more antennas for wirelessly communicating with, for example, other devices, websites, and the sensor device 106). Communication between the stimulator 102 and other devices can be performed via, e.g., a network (which can include the Internet, public and private intranet, a local or extended Wi-Fi network, cell towers, the plain old telephone system (POTS), etc.), direct wireless communication, etc. The stimulator 102 can additionally include well-known input components and output components, including, for example, a touch screen, a keypad, speakers, a camera, etc.

In operation, the patient may receive output or instructions from the computing device 104 that are based at least in part on data received at the computing device 104 from the sensor device 106 and/or the stimulator 102. For example, the sensor device 106 may generate a stroke indication based on analysis of data collected via sensing components. The sensor device 106 may then instruct the computing device 104 to output an alert to the patient or another entity. In some embodiments, the alert can both be displayed to the user (e.g., via display of the external device) and can also be transmitted to an appropriate emergency medical response service (e.g., a 9-1-1 call may be placed with location data from the computing device 104 used to direct responders to locate the patient), and/or to other healthcare provider entities or individuals (e.g. a hospital, emergency room, or physician). In some embodiments, embedded circuitry that provides location data (e.g., a GPS unit) can be included within the sensor device 106.

Additionally or alternatively, the computing device 104 may output user prompts which may be used in conjunction with physiological data collection via the sensor device 106. For example, the computing device 104 may instruct the user to perform an action (via the stimulator 102 or other communication means) (e.g., lift an arm, make a facial expression, etc.), and the sensor device 106 may record physiological data while the user performs the requested actions. In some embodiments, the computing device 104 may itself analyze physiological parameters of the patient, for example using a camera integrated with the stimulator 102 or separate from the stimulator 102 to detect facial drooping or other indicia of stroke. In some embodiments, such physiological data collected via the computing device 104 can be combined with data collected via the sensing components and analyzed together to make a stroke determination.

As noted previously, the external computing device(s) can take the form of servers or other computing devices associated with healthcare providers or other entities. The external devices can include a communications link (e.g., components to facilitate wired or wireless communication with other devices either directly or via the network), a memory, and processing circuitry. These external computing devices can collect data recorded by the sensor device 106 and/or the computing device 104. In some embodiments, such data can be anonymized and aggregated to perform large-scale analysis (e.g., using machine-learning techniques or other suitable data analysis techniques) to develop and improve stroke detection algorithms using data collected by a large number of sensor devices 106 associated with a large population of patients. Additionally, the external computing devices may transmit data to the computing device 104 and/or the sensor device 106. For example, an updated algorithm for making stroke determinations may be developed by the external computing devices (e.g., using machine learning or other techniques) and then provided to the sensor device 106, stimulator 102, and/or the computing device 104 via the network, and installed on the recipient device 102/104/106.

### Example Methods

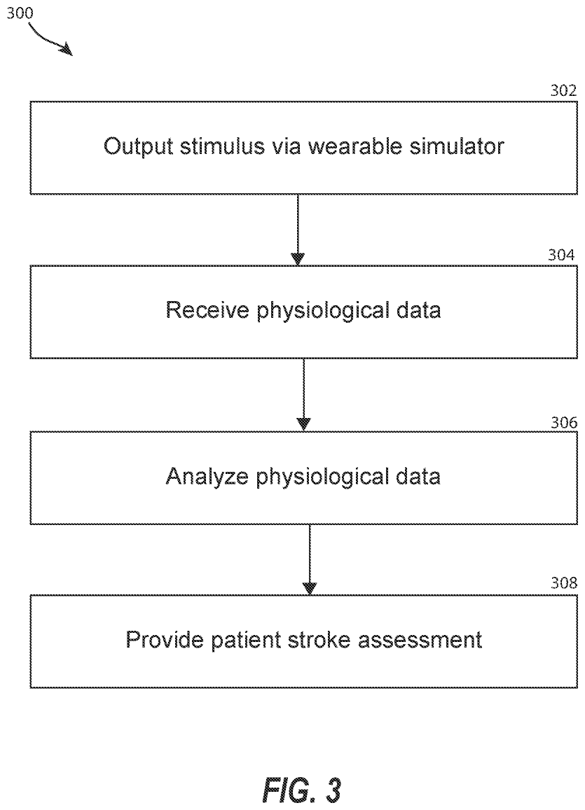

FIG. 3 is a flow diagram of a method 300 for making a stroke assessment. The process 300 can include instructions stored, for example, in the memory (e.g., memory of the system 100 shown in FIG. 1) that are executable by the one or more processors (e.g., the processing circuitry of the system 100 shown in FIG. 1). In some embodiments, portions of the process 300 are performed by one or more hardware components (e.g., the sensing components of the system 100 of FIG. 1). In certain embodiments, portions of the process 300 are performed by a device external to the system of FIG. 1.

As illustrated, the process 300 begins in block 302 with outputting a stimulus to the patient via the wearable stimulator. The wearable stimulator may be the wearable stimulator 102 described above with respect to system 100. The stimulus may be a visual stimulus and/or an audio stimulus. The process 300 continues at block 304 with obtaining sensor data, such as EEG sensor data, via a sensor device disposed on or in the patient. The sensor device may be, for example, the sensor device 106 described above with reference to system 100. The sensor device may include one or more electrodes implanted subcutaneously and/or positioned over the patient's skin. In some embodiments, the sensor data can include electrical signals detected using electrodes of a sensor device 106 as described above with respect to FIG. 1.

In some embodiments, the process includes filtering the EEG sensor data to remove ECG artifacts. Conventionally, EEG data has been obtained via electrodes positioned over the scalp because it is a relatively noise-free location for signal acquisition. Other anatomical locations such as back of the neck have not been used, not because the EEG signal is not present, but because of the noisier environment and band overlap with other physiologic signals such as ECG. However, recent techniques for machine learning/adaptive neural network processing have enhanced the signal extraction capability (e.g., to filter out or reduce the contribution of ECG signals from the EEG signals). One such methodology is described in “ECG Artifact Removal of EEG signal using Adaptive Neural Network” as published in IEEE Xplore 27 May 2019, which is hereby incorporated by reference in its entirety. Similarly, electrical signals associated with muscle activity may also be filtered from the EEG sensor data to remove such artifacts.

In block 306, the physiological data is analyzed and at block 308 a patient stroke assessment is provided. The patient stroke assessment may include, for example, a binary output of stroke condition/non-stroke condition, a probabilistic indication of stroke likelihood, or other output relating to the patient's condition and likelihood of having suffered a stroke. This stroke assessment can be calculated using a classifier model as described elsewhere herein. In addition to providing the patient stroke assessment, information or instructions can also be output to a patient or user. For example, if a stroke is identified in block 308, then the system may provide instructions to route the patient to a comprehensive stroke treatment center or otherwise flag the patient for treatment. In embodiments in which the process 300 is performed while the patient is in an ambulance, the process 300 can output information or instructions to an emergency medical technician (EMT) or other personnel in the rear of the ambulance and/or to the ambulance driver. In some embodiments, the display to the ambulance driver can include navigational information such as a map and instructions to take the patient to a particular hospital or facility with a stroke center.

In some embodiments, prior to, concurrently with, or after providing the stroke assessment in block 308, the method 300 can include triggering an automatic data transmission, for example of a stroke determination which can be output to the patient or another entity (e.g., a call center, emergency response personnel, etc.). A call center may contact the patient or a patient's designated contact to inquire as the patient's status, and/or to confirm a patient stroke. If the patient stroke is confirmed (or if the call center is unable to reach the patient), a 9-1-1 emergency call can be initiated, either manually by call center personnel or automatically.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Unless otherwise indicated, all numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising,” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments. For example, a master-slave configuration could be possible leveraging the well-established pectoral implant location to derive cardiac ECG information and the back-of-head/neck implant location to derive neuro EEG information. These slave devices can be converged into a master device that could be an external smartwatch or smartphone to provide stroke detection capability.

1-70. (canceled)

71. A system for assessing stroke conditions for a patient, the system comprising:
a sensor configured to receive physiological data from the patient;
a wearable stimulator configured to generate a stimulus; and
a computing device communicatively coupled to the sensor and the wearable stimulator, the computing device configured to:
cause the wearable stimulator to output a stimulus configured to trigger a stroke event for the patient;
receive the physiological data from the sensor; and
based on the physiological data, indicate if a stroke event has occurred.

72. The system of claim 71, wherein the stimulus is a visual stimulus and/or an audio stimulus.

73. The system of claim 71, wherein the wearable stimulator is a wearable display.

74. The system of claim 71, wherein the wearable stimulator is a virtual reality (VR) headset.

75. The system of claim 71, wherein the physiological data comprises brain activity data.

76. The system of claim 71, wherein the sensor comprises a plurality of electrodes configured to detect brain activity data.

77. The system of claim 71, wherein the sensor comprises a plurality of electroencephalogram (EEG) electrodes.

78. The system of claim 71, wherein providing the patient stroke assessment includes classifying an identified stroke as ischemic or hemorrhagic.

79. The system of claim 71, wherein providing the patient stroke assessment includes determining whether a patient has suffered a stroke.

80. The system of claim 71, wherein providing the patient stroke assessment includes determining a risk that a patient will suffer a stroke.

81. The system of claim 71, wherein providing the patient stroke assessment includes providing a confidence score associated with a determination of patient stroke.

82. A method for assessing stroke conditions, comprising:
outputting a stimulus to a patient via a wearable stimulator;
receiving physiological data from a sensor configured to obtain the physiological data from the patient;
analyzing the physiological data; and
based on the analysis, providing a patient stroke assessment, wherein the patient stroke assessment is at least one of determining whether a patient has suffered a stroke, classifying an identified stroke as ischemic or hemorrhagic, and/or determining a risk that a patient will suffer a stroke.

83. The method of claim 82, wherein the computing device is configured to output a visual stimulus and/or an audio stimulus via the wearable stimulator, wherein each of the visual stimulus and the audio stimulus is configured to stimulate a stroke event for the patient.

84. The method of claim 82, wherein the stimulator is a wearable display.

85. The method of claim 82, wherein the stimulus is a first stimulus and the method further comprises outputting a second stimulus after the first stimulus based on the analysis.

86. The method of claim 85, wherein the second stimulus is different than the first stimulus.

87. The method of claim 82, wherein the physiological data comprises brain activity data.

88. The method of claim 82, wherein the sensor comprises a plurality of electrodes configured to detect brain activity data.

89. The method of claim 82, wherein the sensor comprises a plurality of electroencephalogram (EEG) electrodes.

90. The method of claim 82, wherein providing the patient stroke assessment includes providing a confidence score associated with a determination of patient stroke.

91. The method of claim 82, wherein providing the patient stroke assessment comprises transmitting an alert to an emergency healthcare provider.
